(19) United States
(12) Patent Application Publication
(10) Pub. No.: US 2010/0305534 A1
(43) Pub. Date: Dec. 2, 2010

(54) REUSABLE ONE-SIDE ABSORPTION CUSHION

(76) Inventor: Jason Yang, Taichung City (TW)

Correspondence Address: SINORICA, LLC, 2275 Research Blvd., Suite 500, ROCKVILLE, MD, 20850, US

(21) Appl. No.: 12/476,901

(22) Filed: Jun. 2, 2009

(51) Int. Cl. A61F 13/15

(52) U.S. Cl. 604/367; 604/378; 604/385.14

(57) ABSTRACT

A reusable one-side absorption cushion mainly includes an absorption layer made of a woven fiber, and a waterproof layer made of a waterproof material for being adhered to one side of the absorption layer. By attaching the reusable one-side absorption cushion to a conventional diaper, the urine of a baby can be absorbed by the absorption layer and stopped by the waterproof layer. If the urine doesn't permeate the circumference of the reusable one-side absorption cushion, it is only necessary to replace the reusable one-side absorption cushion with a new one, leaving the conventional diaper to be continuously uses. And, the used reusable one-side absorption cushion can be reused after being cleaned up thoroughly, so as to consume less conventional diapers to achieve purposes of energy saving and waste reduction.

### BACKGROUND OF THE INVENTION

1. Field of the Invention

This invention relates to a reusable one-side absorption cushion with a conventional diaper while using.

2. Description of the Prior Art

Diapers have been widely used on babies and their absorption and comfort are two main properties that are commercially emphasized. While diapers are pretty convenient in using, however, they only can be used once; meanwhile, they have to be discarded and replaced with a new one after being contaminated Therefore, conventional diapers cause not only source waste and treatment problems waste, but it is also a great economic burden for parent when using them.

### SUMMARY OF THE INVENTION

The object of this invention is to offer a reusable one-side absorption cushion with a conventional diaper in using. The reusable one-side absorption cushion mainly includes an absorption layer and a waterproof layer. The absorption layer is made of a woven fiber with a high property of water absorption, able to be easily cleaned up and rewashed with water. The waterproof layer is made of a waterproof material to prevent liquid spreading through the conventional diaper.

From this invention, it has provided the reusable one-side absorption cushion which can be used with the conventional diaper. The combination of the absorption layer and the waterproof layer, the reusable one-side absorption cushion can absorb urine and prevent it from permeating in a single direction. When baby stools, the urine will be absorbed by the absorption layer immediately, but if the urine can not be totally absorbed, it will permeate through the molecule of the fibers of the absorption layer toward to the waterproof layer. Then the urine will be blocked from further permeating into the conventional diaper while coming to the waterproof layer. If the liquid has not permeated through the circumference of the reusable one-side absorption cushion, we can only replace the reusable one-side absorption cushion, and the conventional diaper still can be continuously used. Therefore, the used reusable one-side absorption cushion can be reused after being cleaned up, so as to consume less conventional diapers to achieve purposes of energy saving and waste reduction.

### BRIEF DESCRIPTION OF DRAWINGS

This invention is easier to understand by referring to the accompanying drawings, wherein:

FIG. 1 is a perspective view of a first preferred embodiment of a reusable one-side absorption cushion in the present invention;

FIG. 2 is a cross-sectional view of the first preferred embodiment of a reusable one-side absorption cushion in the present invention;

FIG. 3 is a perspective view of the first preferred embodiment of a reusable one-side absorption cushion in the present invention, showing it being used with a conventional diaper

FIG. 4 is an illustrating perspective view of the first preferred embodiment in the present invention, showing how it absorbs water and prevents it from permeating through;

FIG. 5 is a cross-sectional view of a second preferred embodiment of a reusable one-side absorption cushion in the present invention; and

FIG. 6 is a cross-sectional view of a third preferred embodiment of a reusable one-side absorption cushion in the present invention.

### DETAILED DESCRIPTION OF THE PREFERRED EMBODIMENT

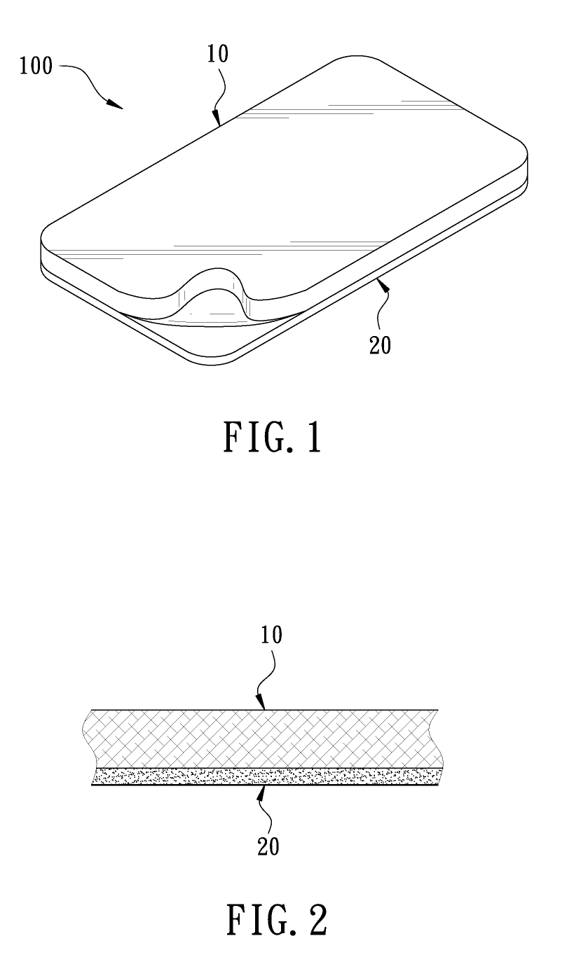

As shown in FIGS. 1 and 2, the first preferred embodiment of a reusable one-side absorption cushion 100 in the present invention includes an absorption layer 10 and a waterproof layer 20.

The absorption layer 10 is made of a natural or it also can be synthetic woven fiber with a high property of water absorption

The waterproof layer 20 is attached to one side of the absorption layer 10, made of a waterproof material for preventing water from permeating through. In the embodiment, the waterproof layer 20 is made of a high polymer film, adhered to one side of the absorption layer 10.

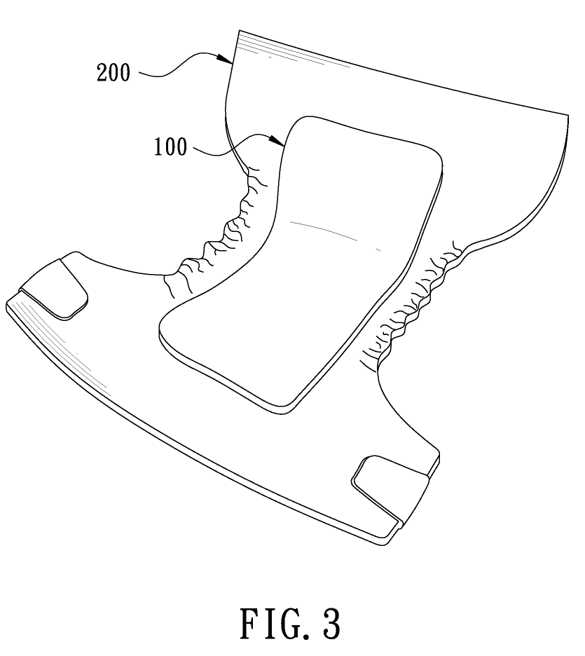

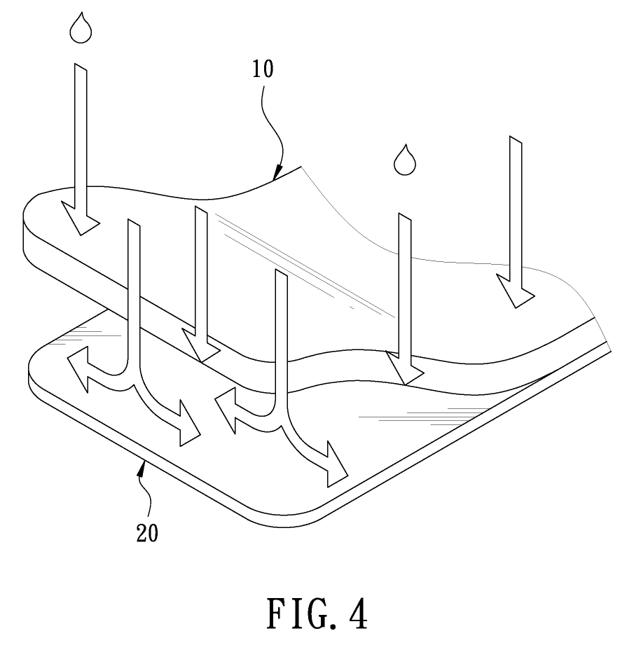

In addition, as shown in FIGS. 3 and 4, while using, the reusable one-side absorption cushion 100 comes with a conventional diaper 200. The reusable one-side absorption cushion 100 is properly laid on the surface of the conventional diaper 200 to attach to baby's skin, with the waterproof layer 20 attaching to the conventional diaper 200, so that the absorption layer 10 can attach to a baby's body to enable the reusable one-side absorption cushion 100 to absorb urine and prevent it from permeating in a single direction. Referring to FIG. 4 again, when baby stools, its liquid can be primarily absorbed by the absorption layer 10, permeating through molecule of the fibers of the absorption layer 10 toward the waterproof layer 20 as shown by the arrows. The liquid will be blocked from further permeating into the conventional diaper 200 while it comes out. If the liquid has totally been absorbed by the absorption layer and insulated by the waterproof layer 20, not permeating through the circumference of the reusable one-side absorption cushion 100, it is only necessary to replace the reusable one-side absorption cushion 100 and the conventional diaper 200 can still be used continuously. Therefore, the used reusable one-side absorption cushion 100 can be reused after being cleaned up thoroughly.

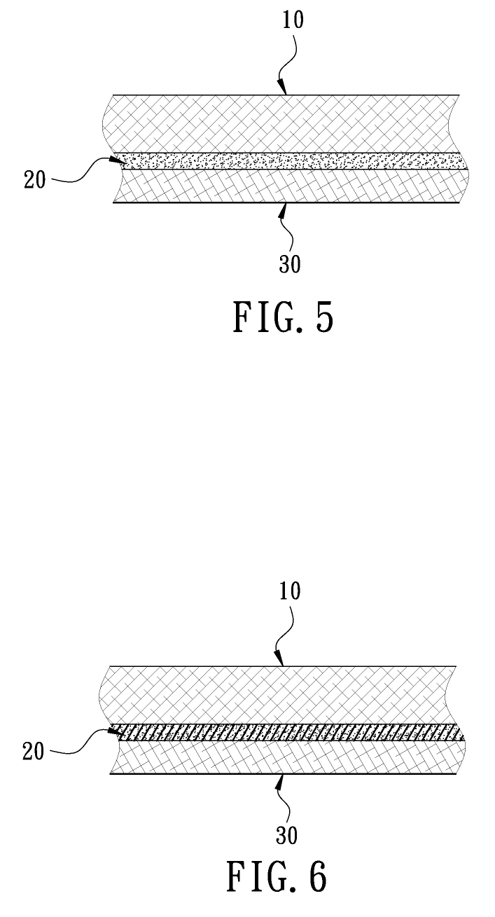

As shown in FIG. 5, a second preferred embodiment of a reusable one-side absorption cushion 100 in the present invention has the same structure and the same effects as the first embodiment does, except that an extra fiber-woven layer 30 is adhered to one side of the waterproof layer 20. The absorption layer 10 is adhered to or sewn with the other side of the waterproof layer 20.

Next, as shown in FIG. 6, a third preferred embodiment of a reusable one-side absorption cushion 100 in the present invention has the same structure and the same effects as the first embodiment does, except that the waterproof layer 20 is a layer of waterproof resin able to prevent liquid from penetrating through it. The fiber-woven layer 30 is adhered to or sewn with the other side of the waterproof resin layer 20.

While the preferred embodiment of the invention has been described above, it will be recognized and understood that various modifications may be made therein and the appended claims are intended to cover all such modifications that may fall within the spirit and scope of the invention.

What is claimed is:

1. A reusable one-side absorption cushion comprising:
an absorption layer made of a woven fiber with a high property of water absorption; and
a waterproof layer attached to one side of absorption layer and made of a waterproof material for preventing a liquid from permeating through it.

2. The reusable one-side absorption cushion as claimed in claim 1, wherein said absorption layer is made of a natural fiber.

3. The reusable one-side absorption cushion as claimed in claim 1, wherein said absorption layer is made of a synthetic fiber.

4. The reusable one-side absorption cushion as claimed in claim 1, wherein said waterproof layer is made of a high polymer film.

5. The reusable one-side absorption cushion as claimed in claim 4, wherein a fiber-woven layer is further adhered to one side of said waterproof layer made of a high polymer film.

6. The reusable one-side absorption cushion as claimed in claim 1, wherein said waterproof layer is coated with a layer of waterproof resin having one side adhered to a fiber-woven layer.
